(19) United States
(12) Patent Application Publication
(10) Pub. No.: US 2025/0302498 A1
(43) Pub. Date: Oct. 2, 2025

(54) INSERTION FORCEPS

(71) Applicant: iMacular Regeneration LLC, Rochester, MN (US)

(72) Inventors: Timothy W. Olsen, Rochester, MN (US); John Christian Love, San Diego, CA (US); Mikhail Borisovich Tikh, St. Louis Park, MN (US)

(21) Appl. No.: 18/620,376

(22) Filed: Mar. 28, 2024

(51) Int. Cl. A61B 17/28; A61B 17/00

(52) U.S. Cl. CPC A61B 17/282; A61B 17/2841; A61B 2017/00367; A61B 2017/00526; A61B 2017/2845

(57) ABSTRACT

An example device can include a tubular segment. The tubular segment can have a first end face, a second end, a center axis, and a sidewall. The first end face is disposed at the first end and is contiguous with the second end and aligned on the center axis. The sidewall has an aperture disposed proximate to the first end face and between the first end face and the second end. A shaft of the device is configured for axial movement relative to the tubular segment and within a portion of the tubular segment. The shaft is configured to exert a clamping force between a distal end of the shaft and a portion of the first end face.

### TECHNICAL FIELD

This document pertains generally, but not by way of limitation, to a hand-operated tool for grasping and manipulating an object for use in a surgical procedure.

### BACKGROUND

Forceps typically used for surgical procedures can include a pair of jaws coupled at a pivot to a pair of corresponding handles. Manipulation of the handles controls opening and closing of the jaws in the manner of a pair of scissors or a pair of pliers. Such familiar tools are convenient for grasping and cutting purposes when used with relatively large objects, however precision can be complicated. The pivot location can be adjusted to provide finer cutting or grasping action but this requires a greater travel distance for the handle.

U.S. Pat. No. 11,160,935 refers to a reusable instrument handle with single-use tip that may include an instrument tip and an instrument handle.

### SUMMARY

The present inventor has recognized, among other things, that a problem of fine control of grasping and manipulating in a surgical setting requires a jaw configured to travel at an appropriate scale and with good operator control.

The present subject matter can help provide a solution to this problem, such as by arranging a shaft to travel axially within a sheath (or a cannula or sleeve) and configured to exert a clamping force between an end of the shaft and a jaw portion of the sheath. The jaw portion can be provided by a sidewall cut-out segment of the sheath disposed near an operating end. An actuator, such as a hand-controlled squeeze-bulb, can be affixed to the sheath and shaft and provide good control of the clamping action.

An example device can include a tubular segment. The tubular segment can have a first end face, a second end, a center axis, and a sidewall. The first end face is disposed at the first end and is contiguous with the second end and aligned on the center axis. The sidewall has an aperture disposed proximate to the first end face and between the first end face and the second end. A shaft of the device is configured for axial movement relative to the tubular segment and within a portion of the tubular segment. The shaft is configured to exert a clamping force between a distal end of the shaft and a portion of the first end face.

In one example, the first end face is cantilevered to the sidewall. The cantilevered configuration can include an aperture in the sidewall. The first end face can be affixed to a segment of the sidewall.

In one example, a projection is affixed to a portion of the first end face. The projection can have a linear dimension aligned with a segment of the sidewall. The projection can include a cross section configured to control rotary motion about the center axis. The projection can include a cross section which is round, triangular, square or other shape.

In one example, the first end face is oblique relative to the center axis. In one example, the shaft has an end that is oblique relative to the center axis. In one example, the shaft end is rounded.

In one example, the aperture in the sidewall includes an edge having a non-perpendicular angle relative to the center axis. The non-perpendicular angle provides clearance for receiving and manipulating the tab element of the object.

In one example, the proximal end of the shaft is configured to engage with a manual actuator tool. In one example, the tubular segment second end is configured to engage with a manual actuator tool. The manual actuator tool can include a squeeze bulb or a handled instrument with a pivoting joint.

One example includes a device having a shaft within a sheath. The shaft is configured for axial movement relative to the sheath. The device can also include a foot affixed to the sheath. A portion of the foot is aligned with a longitudinal axis of the shaft. The sheath can include a throat portion adjacent the foot.

In one example, the throat portion includes an aperture in a sidewall of the sheath.

In one example, the foot includes a clamp face aligned oblique with the longitudinal axis.

In one example, shaft is configured to exert a compressive force in a direction of the clamp face.

In one example, the foot includes an aperture aligned with the longitudinal axis.

In one example, the aperture in the foot has an aperture diameter substantially equal to an inner diameter of the sheath.

In one example, the foot includes a tooth aligned parallel with the longitudinal axis.

In one example, the foot includes an indexing structure to enable controllable rotation of an object engaged with the foot and the shaft. The indexing structure can include a sidewall or a polygonal stud. In one example, the shaft includes a conical end profile or a rounded end profile.

One example of the present subject matter includes a method. The method can include positioning a shaft in a sheath. The method can include affixing a first jaw to an end of the sheath. The method can include providing a retainer on a jaw face of the first jaw. The method can include configuring the shaft for axial movement relative to the sheath.

The method can include affixing the first jaw to an end of the sheath and can include forming a throat region adjacent an end of the sheath.

In one example, providing the retainer on the jaw face includes forming a stud.

In one example, configuring the shaft for axial movement includes affixing a manual actuator to the shaft and the sheath.

Each of these non-limiting examples can stand on its own, or can be combined in various permutations or combinations with one or more of the other examples.

This overview is intended to provide an overview of subject matter of the present patent application. It is not intended to provide an exclusive or exhaustive explanation of the invention. The detailed description is included to provide further information about the present patent application.

### BRIEF DESCRIPTION OF THE DRAWINGS

In the drawings, which are not necessarily drawn to scale, like numerals may describe similar components in different views. Like numerals having different letter suffixes may represent different instances of similar components. The drawings illustrate generally, by way of example, but not by way of limitation, various embodiments discussed in the present document.

FIG. 1A illustrates a view of a device coupled to an object and coupled to an actuator, according to one example.

FIG. 1B illustrates a view of a device coupled to an object, according to one example.

FIG. 2A illustrates a view of a device, according to one example.

FIGS. 2B and 2C illustrate views of a device and an object, according to one example.

FIGS. 3A and 3B illustrate views of a device, according to one example.

FIGS. 4A, 4B, and 4C illustrate views of a device and an object, according to one example.

FIG. 4D illustrates a section view of a device and an object, according to one example.

FIG. 5 illustrates a view of a device and an object, according to one example.

FIG. 6 illustrates a view of a section view of an actuator, according to one example.

FIG. 7 illustrates a flow chart of a method, according to one example.

### DETAILED DESCRIPTION

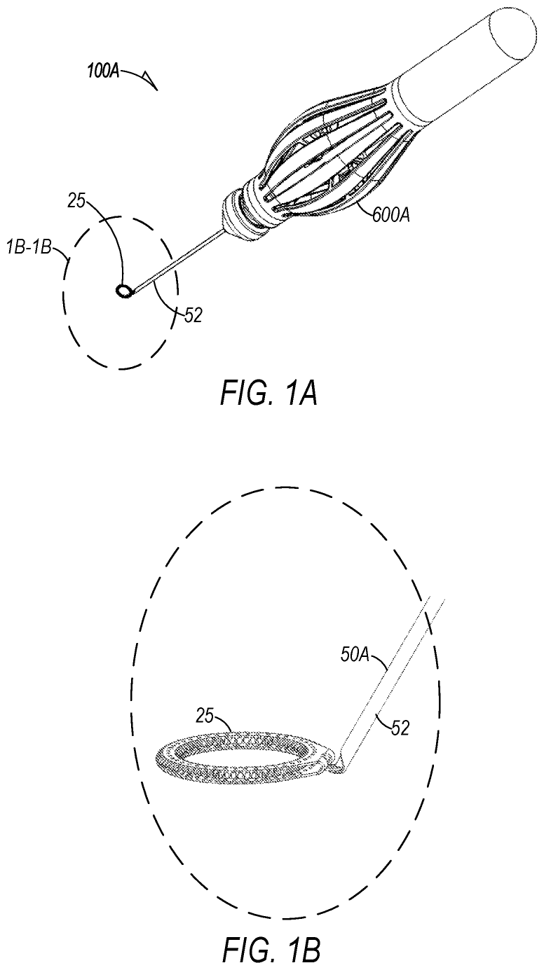

FIG. 1A and FIG. 1B illustrate views of a system 100A including device 52 coupled to object 25 and coupled to actuator 600A, according to one example. FIG. 1A depicts an overview and FIG. 1B depicts selected elements identified at view 1B-1B of FIG. 1A. System 100A includes an insertion forceps, here represented by device 52. Object 25 can include a surgically implanted component having stacked rings, or other structure. In this example, object 25 includes a tab or other element that can be grasped and manipulated by device 52. As depicted in FIG. 1B, object 25 includes a ring-shaped element lying in a plane that is oriented at an oblique angle relative to a longitudinal axis of device 52. Device 52 includes a distal end (shown in FIG. 1B) and a proximal end associated with actuator 600A. Device 52 includes sheath 50A.

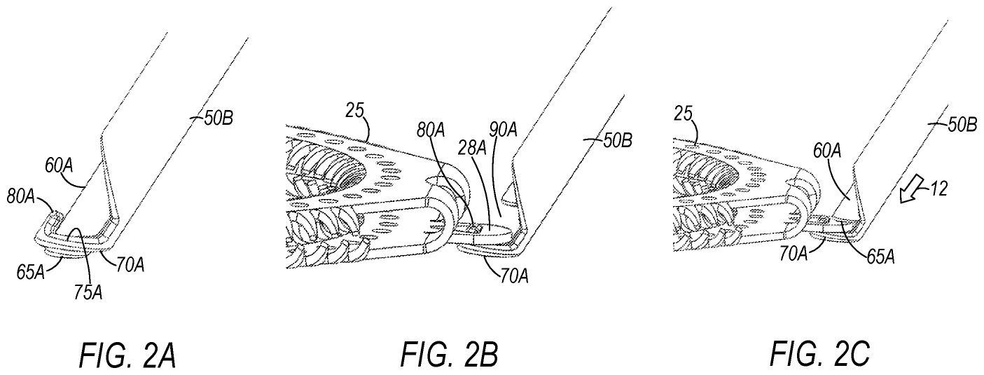

FIG. 2A, 2B, and 2C illustrate views of a device including sheath 50B, according to one example. Sheath 50B includes a sleeve or a tube having a longitudinal axis aligned with arrow 12. Shaft 60A is configured for axial displacement within a lumen of sheath 50B. Throat portion 90A (FIG. 2B), can be configured as an aperture formed in a sidewall of sheath 50B. A portion of shaft 60A is visible in the views of FIG. 2A and FIG. 2C. FIG. 2A also depicts foot 70A affixed to a distal end of sheath 50B. Foot 70A includes a jaw face 75A and includes projection 80A. Projection 80A and jaw face 75A is configured to engage with tab 28A of object 25. In the example here, projection 80A is positioned eccentric relative to the axis of the sheath 50B.

In FIG. 2A, shaft 60A is depicted in a manner wherein shaft end 65A has penetrated an aperture in foot 70A. In FIG. 2B, shaft 60A is depicted in a manner showing shaft 60A substantially retracted within a lumen of sheath 50B. In addition, FIG. 2B depicts projection 80A engaged with a corresponding aperture of tab 28A affixed to object 25. In FIG. 2C, shaft 60A is depicted in a manner abutting tab 28A and positioned to exert a clamping force on tab 28A between end 65A (of shaft 60A) and foot 70A.

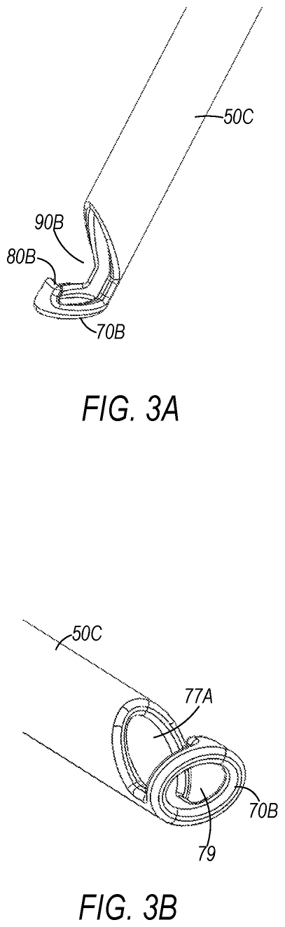

FIGS. 3A and 3B illustrate views of device including sheath 50C, according to one example. In FIG. 3A, a distal end of sheath 50C includes foot 70B. Foot 70B includes projection 80B. In this example, projection 80B can be viewed as a segment of an extended sidewall of sheath 50C. As seen in FIG. 3B, foot 70B includes aperture 79 and sheath 50C includes lumen 77A. The example in FIGS. 3A and 3B can be fabricated by removing material from a metallic tube segment, and as shown, the edges are deburred and rounded by a material removing process such as electropolishing.

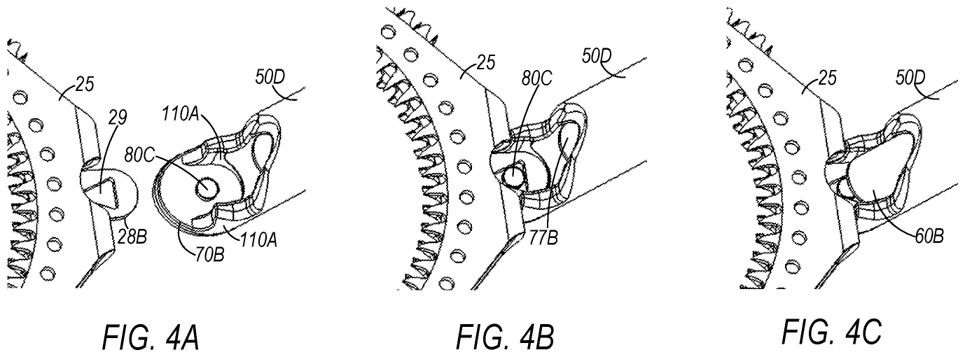

FIGS. 4A, 4B, and 4C illustrate views of a device and object 25, according to one example. FIG. 4A depicts a distal end of sheath 50D including indexing structure 110A flanking projection 80C. Indexing structure 110A can include a portion raised above a jaw face of foot 70B. Indexing structure 110A can engage with one or more sidewalls, or other corresponding structure, of tab 28B affixed to object 25. Projection 80C is disposed on a jaw face of foot 70B and is configured to align with aperture 29 of tab 28B. In the example shown, projection 80C is configured as a stud having a round section, however, in other examples, projection 80C is configured to engage with an aperture of tab 28B in a manner to allow controlled movement of object 25 by manipulating sheath 50D. In the example shown, aperture 29 includes a triangular shaped hole and in one example, projection 80C includes a corresponding shape (such as a triangle) to provide positive engagement without resort to indexing structure 110A. In the example here, projection 80C is positioned substantially aligned with the axis of the sheath 50D.

FIG. 4B illustrates an example in which lumen 77B is aligned on an axis of sheath 50D. In the example, object 25 is engaged with both indexing structure 110A and with projection 80C associated with foot 70B. FIG. 4C illustrates shaft 60B having an axial position to exert a clamping force on tab 28B between a shaft end and a jaw face of foot 70B.

FIG. 4D illustrates a section view of a device and object 25, according to one example. In this view, sheath 50E and shaft 60C are shown in section. Tab 28C is engaged with both indexing structure 110B (shown in the background) and projection 80C (shown in the foreground). This view also illustrates shaft end 65B having a conical configuration. The conical configuration is aligned with the jaw face of foot 70B, and in this example, the jaw face and the shaft end are configured to exert a clamping force that holds object 25 at an oblique angle.

In one example, a projection is provided on shaft end (such as end 65B in FIG. 4D). In this example, the jaw face can be featureless and with axial movement of the shaft (such as shaft 60C in FIG. 4D), the projection engages or disengages with an aperture of a tab.

In one example, the indexing structure (such as indexing structure 110B in FIG. 4D) includes a cantilevered projection coupled to shaft 60C and arranged to extend substantially radial with respect to the axis of shaft 60C. The cantilevered projection can be configured to engage the object 25B and maintain alignment as between object 25B and shaft 60C. In one example, a pair of cantilevered projections can be configured to engage with planar structures of object 25B. As shown in FIGS. 2B and 2C, object 25 can include an upper planar structure and a lower planar structure coupled together at a joint. A first cantilevered projection can engage with object 25 on a first side of the joint (such as at the lower portion of the joint visible in FIG. 4A) and a second cantilevered projection can engage with object 25 on a second side of the joint (such as at the upper portion of the joint visible in FIG. 4A). Other arrangements and structure can be configured to provide an indexing structure in which object 25 is held at a fixed radial alignment with respect to an axis of shaft 60C (FIG. 4D).

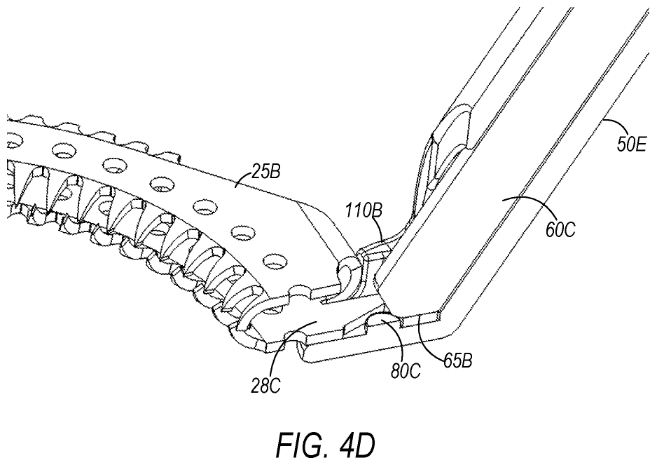

FIG. 5 illustrates a view of a device and object 25, according to one example. In this view, the longitudinal axis of sheath 50F and the planar axis of object 25 are oriented at oblique angle A. In one example, angle A can be substantially 120 degrees.

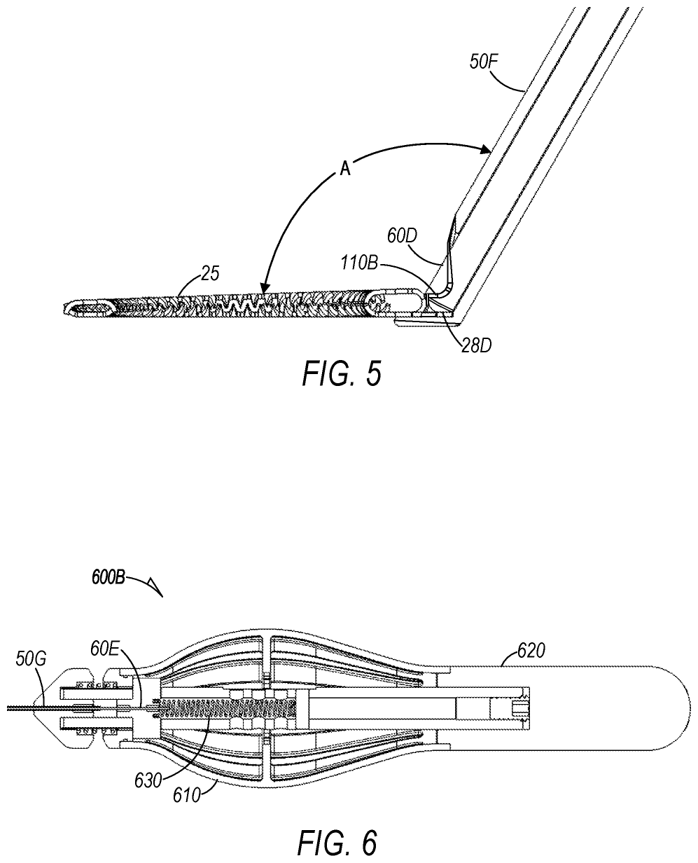

FIG. 6 illustrates a view of a section view of actuator 600B, according to one example. Actuator 600B is shown in partial cross section and includes bulb 610 having a plurality of elastic segments affixed at either end. Bulb 610 can be collapsed by exertion of a manually applied grasping force.

Actuator 600 can be configured to operate with positive action or configured to operate with negative action.

In a positive action configuration, a manually applied squeezing force on bulb 610 provides a positive force on shaft 60E to exert a clamping force between the shaft end and a jaw face and when the bulb 610 is relaxed (that is, not squeezed), shaft 60E is moved in a direction away from the jaw face.

In a negative action configuration, a manually applied squeezing force on bulb 610 moves shaft 60E in a direction away from the jaw face and when the bulb 610 is relaxed (that is, not squeezed), shaft 60E exerts a clamping force between the shaft end and a jaw face.

System configurations and device operation can be summarized by the following table.

| | POSITIVE | NEGATIVE |
|---|---|---|
| | CONFIGURATION | CONFIGURATION |
| Squeeze bulb | clamped | released |
| Relax bulb | released | clamped |

As such, in one configuration, a manually applied squeeze force overcomes spring 630 and induces axial movement of shaft 60E relative to sheath 50G. A user can grasp actuator 600B, via handle 620, and either squeeze or release bulb 610 to control axial movement of shaft 60E relative to sheath 50G.

In one example of a negative configuration, a squeezing force exerted on bulb 610 withdraws shaft 60E relative to sheath 50G in a manner to allow clearance at a distal end between shaft end and the jaw face. Conversely, when released, or not squeezed, spring 630 exerts a force to eject shaft 60E from sheath 50G in a manner to exert a clamping force between shaft end and jaw face. A positive configuration can include actions as noted in the table here.

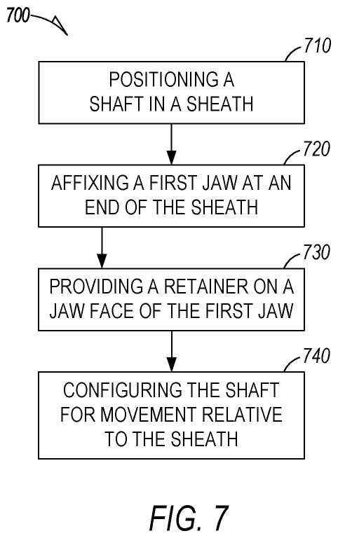

FIG. 7 illustrates a flow chart of method 700, according to one example. Method 700 refers to a method of manufacturing a device as described herein.

At 710, method 700 includes positioning a shaft in a sheath. The shaft is configured to move in an axial direction with respect to the sheath. At a distal end, the shaft and the sheath are accessible and configured to engage an object suitable for use in a surgical procedure.

At 720, method 700 includes affixing a first jaw at an end of the sheath. The first jaw can be associated with a foot portion. The foot portion remains in a fixed position and alignment relative to the sheath. The shaft, travelling within the sheath, can be viewed as dynamic component of a clamp. Affixing a first jaw can include forming an aperture in a sidewall of a sheath in which a remaining portion of the sidewall forms clamping face. In one example, forming a jaw can include bonding a jaw portion to a segment of a sheath. Bonding can include welding, soldering, adhesively joining or other manner of coupling. In one example forming a first jaw can include performing an additive manufacturing process or performing a subtractive manufacturing process.

At 730, method 700 includes providing a projection, or retainer, on a jaw face. The projection can be configured to engage with an element of the object to be grasped. The projection can be formed by performing an additive manufacturing process or performing a subtractive manufacturing process.

At 740, method 700 includes configuring the shaft for movement relative to the sheath. Relative movement in an axial direction can exert a clamping force between a stationary component and a dynamic component. In one example, configuring the shaft for movement relative to the sheath can include coupling an actuator to a sheath and shaft. The actuator can include a manually operated instrument having a pivoting joint or can include a trigger-actuated device or a squeeze-bulb actuated device.

### Various Notes

The present subject matter can be fabricated of tubular stock including stainless steel, a polymer, carbon fibers, or other materials.

An example of the present subject matter can be used in a surgical procedure for implanting a tissue graft in, for example, an eye. For example, a ring-like structure can provide support for a tissue graft. The ring-like structure can be grasped and manipulated by an example of the present subject matter. In one example, the structure can be manipulated to carry a graft to a surgical site.

The above description includes references to the accompanying drawings, which form a part of the detailed description. The drawings show, by way of illustration, specific embodiments in which the invention can be practiced. These embodiments are also referred to herein as “examples.” Such examples can include elements in addition to those shown or described. However, the present inventors also contemplate examples in which only those elements shown or described are provided. Moreover, the present inventors also contemplate examples using any combination or permutation of those elements shown or described (or one or more aspects thereof), either with respect to a particular example (or one or more aspects thereof), or with respect to other examples (or one or more aspects thereof) shown or described herein.

In the event of inconsistent usages between this document and any documents so incorporated by reference, the usage in this document controls.

In this document, the terms “a” or “an” are used, as is common in patent documents, to include one or more than one, independent of any other instances or usages of “at least one” or “one or more.” In this document, the term “or” is used to refer to a nonexclusive or, such that “A or B” includes “A but not B,” “B but not A,” and “A and B,” unless otherwise indicated. In this document, the terms “including” and “in which” are used as the plain-English equivalents of the respective terms “comprising” and “wherein.” Also, in the following claims, the terms “including” and “comprising” are open-ended, that is, a system, device, article, composition, formulation, or process that includes elements in addition to those listed after such a term in a claim are still deemed to fall within the scope of that claim. Moreover, in the following claims, the terms “first,” “second,” and “third,” etc. are used merely as labels, and are not intended to impose numerical requirements on their objects.

Geometric terms, such as “parallel”, “perpendicular”, “round”, or “square”, are not intended to require absolute mathematical precision, unless the context indicates otherwise. Instead, such geometric terms allow for variations due to manufacturing or equivalent functions. For example, if an element is described as “round” or “generally round,” a component that is not precisely circular (e.g., one that is slightly oblong or is a many-sided polygon) is still encompassed by this description.

The above description is intended to be illustrative, and not restrictive. For example, the above-described examples (or one or more aspects thereof) may be used in combination with each other. Other embodiments can be used, such as by one of ordinary skill in the art upon reviewing the above description. The Abstract is provided to allow the reader to quickly ascertain the nature of the technical disclosure. It is submitted with the understanding that it will not be used to interpret or limit the scope or meaning of the claims. Also, in the above Detailed Description, various features may be grouped together to streamline the disclosure. This should not be interpreted as intending that an unclaimed disclosed feature is essential to any claim. Rather, inventive subject matter may lie in less than all features of a particular disclosed embodiment. Thus, the following claims are hereby incorporated into the Detailed Description as examples or embodiments, with each claim standing on its own as a separate embodiment, and it is contemplated that such embodiments can be combined with each other in various combinations or permutations. The scope of the invention should be determined with reference to the appended claims, along with the full scope of equivalents to which such claims are entitled.

The claimed invention is:

1. A device comprising:
a tubular segment having a first end face, a second end, a center axis, and a sidewall, the first end face contiguous with the second end and aligned on the center axis, the sidewall having an aperture disposed proximate to the first end face and between the first end face and the second end; and
a shaft configured for axial movement relative to the tubular segment and within a portion of the tubular segment, the shaft configured to exert a clamping force between a distal end of the shaft and a portion of the first end face.

2. The device of claim 1 wherein the first end face is cantilevered to the sidewall.

3. The device of claim 1 further including a projection affixed to a portion of the first end face.

4. The device of claim 2 wherein the projection includes a linear dimension aligned with a segment of the sidewall.

5. The device of claim 2 wherein the projection includes a cross section configured to control rotary motion about the center axis.

6. The device of claim 1 wherein the first end face is oblique relative to the center axis.

7. The device of claim 1 wherein the shaft end is oblique relative to the center axis.

8. The device of claim 1 wherein the shaft end is rounded.

9. The device of claim 1 wherein the aperture includes an edge having a non-perpendicular angle relative to the center axis.

10. The device of claim 1 wherein a proximal end of the shaft is configured to engage with a manual actuator tool.

11. The device of claim 1 wherein the tubular segment second end is configured to engage with a manual actuator tool.

12. A device comprising:
a shaft within a sheath, wherein the shaft is configured for axial movement relative to the sheath; and
a foot affixed to the sheath, wherein a portion of the foot is aligned with a longitudinal axis of the shaft, and wherein the sheath includes a throat portion adjacent the foot.

13. The device of claim 12 wherein the throat portion includes an aperture in a sidewall of the sheath.

14. The device of claim 12 wherein the foot includes a clamp face aligned oblique with the longitudinal axis.

15. The device of claim 14 wherein the shaft is configured to exert a compressive force in a direction of the clamp face.

16. The device of claim 12 wherein the foot includes an aperture aligned with the longitudinal axis.

17. The device of claim 16 wherein the aperture has an aperture diameter substantially equal to an inner diameter of the sheath.

18. The device of claim 12 wherein the foot includes a tooth aligned parallel with the longitudinal axis.

19. The device of claim 12 wherein the foot includes an indexing structure to enable controllable rotation of an object engaged with the foot and the shaft.

20. The device of claim 19 wherein the indexing structure includes a sidewall or a polygonal stud.

21. The device of claim 12 wherein the shaft includes a conical end profile or a rounded end profile.

22. A method comprising:
positioning a shaft in a sheath;
affixing a first jaw to an end of the sheath;
providing a retainer on a jaw face of the first jaw; and
configuring the shaft for axial movement relative to the sheath.

23. The method of claim 22 wherein affixing the first jaw to an end of the sheath includes forming a throat region adjacent an end of the sheath.

24. The method of claim 22 wherein providing the retainer on the jaw face includes forming a stud.

25. The method of claim 22 wherein configuring the shaft for axial movement includes affixing a manual actuator to the shaft and the sheath.
